(19) United States
(12) Patent Application Publication
(10) Pub. No.: US 2026/0346427 A1
(43) Pub. Date: Nov. 5, 2026

(54) FLEXIBLE WAIST AND ABDOMEN-ASSISTIVE EXOSKELETON WITH DIFFERENTIAL STRUCTURE AND ELASTIC FRAMEWORK

(71) Applicant: DL CRIMSON DYNAMICS TECHNOLOGY CO., LTD., Liaoning (CN)

(72) Inventor: Ke SHANG, Liaoning (CN)

(73) Assignee: DL CRIMSON DYNAMICS TECHNOLOGY CO., LTD., Liaoning (CN)

(21) Appl. No.: 19/486,920

(22) PCT Filed: Apr. 7, 2024
(86) PCT No.: PCT/CN2024/086379

§ 371 Date: Nov. 21, 2025

(30) Foreign Application Priority Data
May 25, 2023 (CN) 202321278706.5

(51) Int. Cl. B25J 9/00; A61F 5/02

(52) U.S. Cl. CPC B25J 9/0006; A61F 5/028

(57) ABSTRACT

A flexible waist and abdomen-assistive exoskeleton with a differential structure and an elastic framework includes the elastic framework. The elastic framework is a frame formed by an upper cross beam, a lower cross beam, a left supporting bar and a right supporting bar. Upper ends and lower ends of bilaterally symmetrical shoulder straps are connected to the elastic framework. The upper cross beam is connected to a first collar, a first woven strap passes through the first collar and has an end connected to the middle of a transverse differential plate. The other end of the first woven strap is connected to an end of a second woven strap by means of a length adjustment device. The other end of the second woven strap is fixed to the lower cross beam. Two ends of the transverse differential plates are respectively connected to vertical elastic pull cords.

### CROSS-REFERENCE TO RELATED APPLICATION

This patent application is a national stage application of International Patent Application No. PCT/CN2024/086379, filed on Apr. 7, 2024, which claims priority of Chinese Patent Application No. 202321278706.5, filed on May 25, 2023, both of which are incorporated by references in their entireties.

### TECHNICAL FIELD

The present disclosure relates to a flexible waist and abdomen-assistive exoskeleton, and in particular to a flexible waist and abdomen-assistive exoskeleton with a differential structure and an elastic framework.

### BACKGROUND

A waist and abdomen-assistive exoskeleton is a wearable device, which can improve the carrying capacity and endurance of a wearer. The utility model patent with Chinese Patent No. CN216442547U discloses a “waist and abdomen-assistive exoskeleton for improving the flexibility of movement and the comfort of wearing”, which is structurally provided with a waistband and symmetrical shoulder straps connected to the waistband. The shoulder straps are located at the upper part of the front part of the waistband and connected to the upper part of a rigid framework structure. The lower part of the rigid framework structure is transversely limited to the side or back of the waistband. The lower end of the rigid framework structure is connected to pull cords, and pull cord fixing members are arranged at the lower parts of the pull cords. The waist and abdomen-assistive exoskeleton is a rigid and flexible integrated exoskeleton, and the assistance effect can be achieved only by applying a pull force to the rigid framework structure by means of the pull cords, such that the rigid framework structure provides to the human body a torque which is opposite to a carrying stress in direction. However, since only the elastic elements (pull cords) provide the assistance effect and are directly connected to leg straps of the exoskeleton, the wearer will repeatedly pull the elastic elements during walking, a resulting resistance affects the natural gait of the human body, and usage burden is increased.

### SUMMARY

The present disclosure aims to solve the above technical problems in the prior art, and to provide a flexible waist and abdomen-assistive exoskeleton with a differential structure and an elastic framework.

The technical solution of the present disclosure is as follows. A flexible waist and abdomen-assistive exoskeleton with a differential structure and an elastic framework is provided, the flexible waist and abdomen-assistive exoskeleton includes an elastic framework; the elastic framework is a frame formed by an upper cross beam, a lower cross beam, a left supporting bar and a right supporting bar, and upper ends and lower ends of bilaterally symmetrical shoulder straps are connected to the elastic framework; the upper cross beam is connected to a first collar, a first woven strap passes through the first collar and has an end connected to a middle of a transverse differential plate, the transverse differential plate is capable of swinging relative to the first woven strap, an other end of the first woven strap is connected to an end of a second woven strap by means of a length adjustment device, and an other end of the second woven strap is fixed to the lower cross beam; two ends of the transverse differential plate are respectively connected to two vertical elastic pull cords, lower ends of the two vertical elastic pull cords are respectively connected to two thigh tying straps, and two limiting guide sleeves fixed at a lower end of the elastic framework are sleeved outside the vertical elastic pull cords.

In some embodiments, a transverse elastic pull cord is connected between the two thigh tying straps.

In some embodiments, the middle of the differential plate is provided with first fixing holes, the first woven strap is fixed by directly passing through the first fixing holes, or a fixing cord passes through the first fixing holes and is then connected to the first woven strap.

In some embodiments, the two ends of the differential plate are provided with second fixing holes, upper ends of the vertical elastic pull cords are connected to the second fixing holes by means of connecting rings.

In some embodiments, second collars are arranged between the lower ends of the vertical elastic pull cords and the two thigh tying straps, the lower ends of the vertical elastic pull cords, two ends of the transverse elastic pull cord and the two thigh tying straps are all connected to the second collars by means of the connecting rings.

In some embodiments, the first collar is connected to the upper cross member by means of a third woven strap, and a chest strap is connected between the bilaterally symmetrical shoulder straps.

In some embodiments, a connecting strap is arranged between the two limiting guide sleeves.

The present disclosure is a flexible waist and abdomen-assistive exoskeleton with a differential structure and an elastic framework, which takes into account the freedom and the better assistance effect required for the movement of the human body. Left and right leg tying straps, the elastic pull cords and the differential plate are connected to form a differential structure. When the left and right legs alternately wiggle back and forth (i.e., the human body walks or runs), the differential structure also swing with leg movements. In this process, the elastic pull cords do not deform, that is, no resistance is generated to the leg movements, thereby avoiding the shortcomings of the impact on the natural gait of the human body and the increase of the use burden in the conventional art. When the human body bends down, the elastic pull cords on the right and left sides are pulled simultaneously, and in this case, the differential structure is in a fixed state and transfers a pull force to achieve the assistance effect. Due to the presence of the differential structure, even if the pull force of the elastic pull cord is increased, the freedom of movement of the human body is not affected. Therefore, the pull force of the elastic pull cords can be increased according to requirements, and the strength of the flexible waist and abdomen-assistive exoskeleton is thus increased.

### BRIEF DESCRIPTION OF THE DRAWINGS

FIG. 1 and FIG. 2 show schematic structural diagrams of an embodiment of the present disclosure.

FIG. 3 and FIG. 4 show schematic structural diagrams illustrating the connection between a differential plate and a first woven strap according to an embodiment of the present disclosure.

### DETAILED DESCRIPTION OF THE EMBODIMENTS

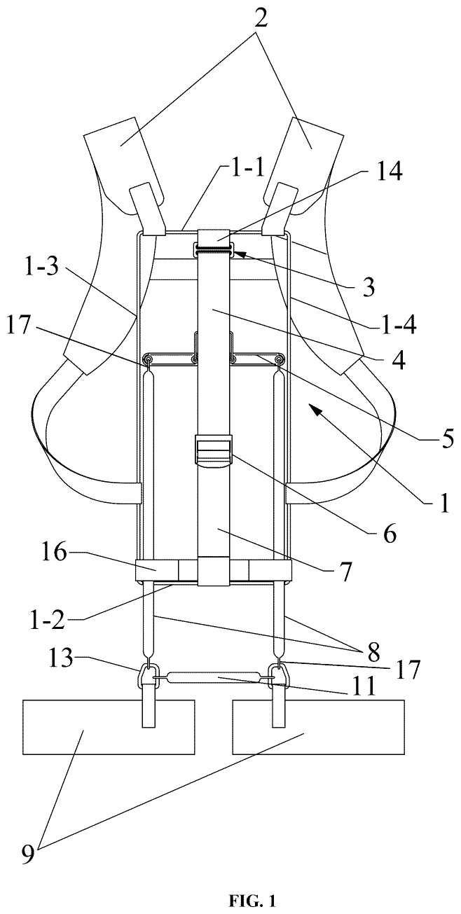

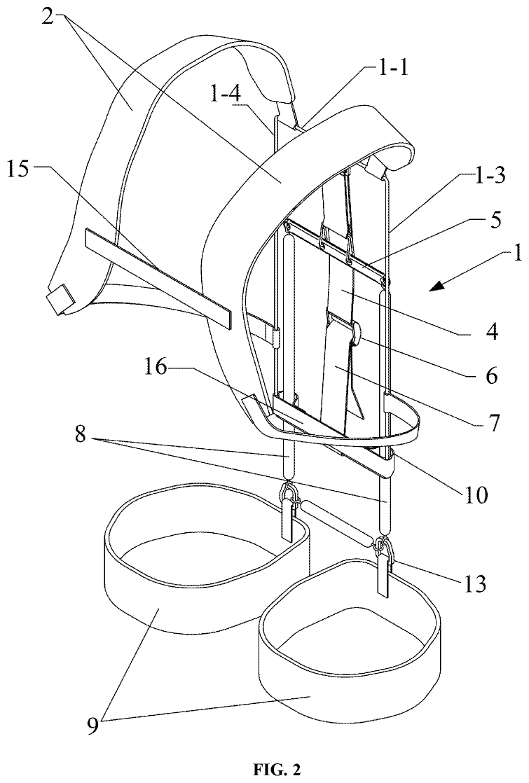

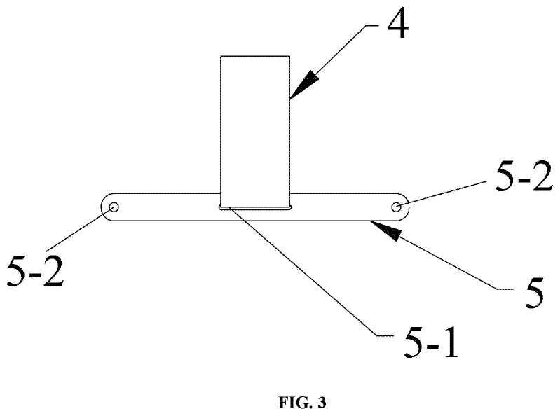

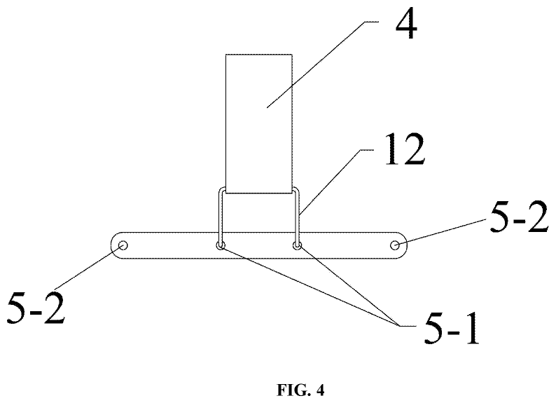

The present disclosure provides a flexible waist and abdomen-assistive exoskeleton with a differential structure and an elastic framework. As shown in FIGS. 1 and 2, the flexible waist and abdomen-assistive exoskeleton is provided with an elastic framework 1, the elastic framework 1 is a rectangular frame formed by an upper cross beam 1-1, a lower cross beam 1-2, a left supporting bar 1-3 and a right supporting bar 1-4, which can be made of elastic materials, for example, a manganese steel, a stainless steel, a composite material, and a titanium alloy. The upper ends and lower ends of bilaterally symmetrical shoulder straps 2 are connected to the elastic framework 1, the upper ends of the two shoulder straps 2 can both be connected to the upper cross beam 1-1, and the lower ends of the two shoulder straps 2 are respectively connected to the lower end of the left supporting bar 1-3 and the lower end of the right supporting bar 1-4. The upper cross beam 1-1 is connected to a first collar 3, the upper cross beam and the first collar can be fixedly connected directly, or by means of a third woven strap 14, the first collar 3 can be a fastener which allows a woven strap passing through the fastener to slide freely, for example, a stair button and a square ring. The first woven strap 4 passes through the first collar 3 and has an end connected to the middle of a transverse differential plate 5. As shown in FIG. 3, the middle of the differential plate 5 is provided with one first fixing hole 5-1, and the first woven strap 4 is fixed by directly passing through the first fixing hole 5-1. Alternatively, as shown in FIG. 4, the middle of the differential plate 5 is provided with two first fixing holes 5-1, and a fixing cord 12 passes through the two first fixing holes 5-1 and is then connected to the first woven strap 4. In brief, it should be ensured that when the left or right end of the transverse differential plate 5 is subjected to an external force, the first woven strap 4 can serve as the fulcrum and the transverse differential plate 5 can swing relative to the first woven strap 4, and when the left and right ends are simultaneously subjected to a balanced external force, the transverse differential plate 5 is in a fixed state. The other end of the first woven strap 4 is connected to an end of a second woven strap 7 by means of a length adjustment device 6, the other end of the second woven strap 7 is fixed to the lower cross beam 1-2, the length adjustment device 6 may be a stair button, a nylon buckle, and so on, as long as the length of the second woven strap 7 can be adjusted. The height adjustment of the differential plate 5 can be achieved by adjusting the length of the second woven strap 7. The two ends of the differential plate 5 are respectively connected to vertical elastic pull cords 8, the lower ends of the two vertical elastic pull cords 8 are respectively connected to thigh tying straps 9. Limiting guide sleeves 10 fixed at the lower end of the elastic framework 1 are sleeved outside the vertical elastic pull cords 8. A transverse elastic pull cord 11 is connected between the two thigh tying straps 9. Both the vertical elastic pull cords 8 and the transverse elastic pull cord 11 can be made of elastic materials, for example, elastic rubber, elastic latex or metal springs. The vertical elastic pull cords 8 are subjected to cooperative limiting of the limiting guide sleeves 10 and the transverse elastic pull cord 11 during stretching, and do not offset too much toward the left side and the right side, thus improving the assistance effect. The cooperative limiting effect on the vertical elastic pull cords 8 can be further optimized by arranging a connecting strap 16 between the limiting guide sleeves 10. The two ends of the differential plate 5 are provided with second fixing holes 5-2, the upper ends of the vertical elastic pull cords 8 are connected to the second fixing holes 5-2 by means of connecting rings 17. Second collars 13 are arranged between the lower ends of the vertical elastic pull cords 8 and the thigh tying straps 9. The lower ends of the vertical elastic pull cords 8, two ends of the transverse elastic pull cord 11 and the thigh tying straps 9 are all connected to the second collars 13 by means of the connecting rings 17. A chest strap 15 is connected between the bilaterally symmetrical shoulder straps 2.

1. A flexible waist and abdomen-assistive exoskeleton with a differential structure and an elastic framework, comprising the elastic framework, wherein the elastic framework is a frame formed by an upper cross beam, a lower cross beam, a left supporting bar and a right supporting bar, and upper ends and lower ends of bilaterally symmetrical shoulder straps are connected to the elastic framework; the upper cross beam is connected to a first collar, a first woven strap passes through the first collar and has an end connected to a middle of a transverse differential plate, the transverse differential plate is capable of swinging relative to the first woven strap, an other end of the first woven strap is connected to an end of a second woven strap by means of a length adjustment device, and an other end of the second woven strap is fixed to the lower cross beam; two ends of the transverse differential plate are respectively connected to two vertical elastic pull cords, lower ends of the two vertical elastic pull cords are respectively connected to two thigh tying straps, and two limiting guide sleeves fixed at a lower end of the elastic framework are sleeved outside the vertical elastic pull cords.

2. The flexible waist and abdomen-assistive exoskeleton with the differential structure and the elastic framework according to claim 1, wherein a transverse elastic pull cord is connected between the two thigh tying straps.

3. The flexible waist and abdomen-assistive exoskeleton with the differential structure and the elastic framework according to claim 1 wherein the middle of the differential plate is provided with first fixing holes, the first woven strap is fixed by directly passing through the first fixing holes, or a fixing cord passes through the first fixing holes and is then connected to the first woven strap.

4. The flexible waist and abdomen-assistive exoskeleton with the differential structure and the elastic framework according to claim 3, wherein the two ends of the differential plate are provided with second fixing holes, upper ends of the vertical elastic pull cords are connected to the second fixing holes by means of connecting rings.

5. The flexible waist and abdomen-assistive exoskeleton with the differential structure and the elastic framework according to claim 4, wherein second collars are arranged between the lower ends of the vertical elastic pull cords and the two thigh tying straps, the lower ends of the vertical elastic pull cords, two ends of the transverse elastic pull cord and the two thigh tying straps are all connected to the second collars by means of the connecting rings.

6. The flexible waist and abdomen-assistive exoskeleton with the differential structure and the elastic framework according to claim 5, wherein the first collar is connected to the upper cross member by means of a third woven strap, and a chest strap is connected between the bilaterally symmetrical shoulder straps.

7. The flexible waist and abdomen-assistive exoskeleton with the differential structure and the elastic framework according to claim 1 wherein a connecting strap is arranged between the two limiting guide sleeves.

8. The flexible waist and abdomen-assistive exoskeleton with the differential structure and the elastic framework according to claim 2, wherein the middle of the differential plate is provided with first fixing holes, the first woven strap is fixed by directly passing through the first fixing holes, or a fixing cord passes through the first fixing holes and is then connected to the first woven strap.

9. The flexible waist and abdomen-assistive exoskeleton with the differential structure and the elastic framework according to claim 8, wherein the two ends of the differential plate are provided with second fixing holes, upper ends of the vertical elastic pull cords are connected to the second fixing holes by means of connecting rings.

10. The flexible waist and abdomen-assistive exoskeleton with the differential structure and the elastic framework according to claim 9, wherein second collars are arranged between the lower ends of the vertical elastic pull cords and the two thigh tying straps, the lower ends of the vertical elastic pull cords, two ends of a transverse elastic pull cord and the two thigh tying straps are all connected to the second collars by means of the connecting rings.

11. The flexible waist and abdomen-assistive exoskeleton with the differential structure and the elastic framework according to claim 10, wherein the first collar is connected to the upper cross member by means of a third woven strap, and a chest strap is connected between the bilaterally symmetrical shoulder straps.

12. The flexible waist and abdomen-assistive exoskeleton with the differential structure and the elastic framework according to claim 2, wherein a connecting strap is arranged between the two limiting guide sleeves.
